(19) United States
(12) Patent Application Publication
(10) Pub. No.: US 2013/0330727 A1
(43) Pub. Date: Dec. 12, 2013

(54) INTRA-TISSUE IN VITRO DIAGNOSIS METHOD FOR DIAGNOSING BRAIN TUMOURS

(71) Applicants: Jean-Paul Issartel, Saint-Egreve (FR); François Berger, Meylan (FR); Elodie Lages, Grenoble (FR); Audrey Guttin, Chassignieu (FR); Michèle El Atifi-Borel, Froges (FR); Hélène Ipas, Arette (FR)

(72) Inventors: Jean-Paul Issartel, Saint-Egreve (FR); François Berger, Meylan (FR); Elodie Lages, Grenoble (FR); Audrey Guttin, Chassignieu (FR); Michèle El Atifi-Borel, Froges (FR); Hélène Ipas, Arette (FR)

(73) Assignees: CENTRE HOSPITALIER UNIVERSITAIRE DE GRENOBLE, Grenoble Cedex 09 (FR); UNIVERSITE JOSEPH FOURIER, Grenoble Cedex 09 (FR)

(21) Appl. No.: 13/992,827

(22) PCT Filed: Dec. 8, 2011
(86) PCT No.: PCT/FR2011/052910

§ 371 Date: Aug. 22, 2013

(30) Foreign Application Priority Data
Dec. 8, 2010 (FR) 1060278

(51) Int. Cl. C12Q 1/68

(52) U.S. Cl. CPC C12Q 1/6886
USPC 435/6.11

(57) ABSTRACT

An in vitro diagnostic method for diagnosing a brain tumour belonging to the group formed by two types of tumours: ODGs and GBMs, and for identifying the type of tumour, the method includes the measurement of at least two ratios of the expression levels of miRNA pairs extracted from a biological sample originating from a patient suspected of presenting one of the above-mentioned brain tumours.

The present invention relates to an in vitro intra-tissue diagnostic method for diagnosing brain tumours, using miRNAs as biomarkers.

Tumours of the central nervous system (CNS) are mostly gliomas (50%), comprising glioblastomas (GBMs) and oligodendrogliomas (ODGs). These tumours are a significant cause of morbidity and mortality and constitute a significant public health problem. In fact, tumours of the CNS are the second greatest cause of death by cancer in children and the third greatest cause in adults (15-34 years). The global incidence of CNS tumours is close to 7,000 new cases discovered each year in France. Lastly, of 6 million deaths per year in the world caused by some form of cancer, 1 to 2% are attributable to tumours of the CNS (data from the World Health Organisation).

Gliomas or glial tumours are classified by the World Health Organisation (WHO) into 4 histological grades, the grade indicating in principle the degree of malignancy. Grade II oligodendrogliomas (ODGs), developed from oligodendrocytes, are low-grade gliomas. By contrast, grade IV glioblastomas (GBMs), developed from astrocytes, are high-grade gliomas. For oligodendrogliomas, the average survival is approximately 7 years, whereas it is only approximately 1 year for glioblastomas after treatment; accurate diagnosis of the nature of the tumour is therefore of decisive importance. Exeresis of the tumour is a way, albeit seemingly radical, for eradicating a tumour although this strategy remains flawed in clinical practice. The ablation of the tumour should be exhaustive, although this is far from being the case because tumours of an infiltrating nature will have “irradiated” pre-tumoral cells in the cerebral regions close to the primitive tumour and also because the limits of the tumour are poorly evaluated during the diagnostic process.

Currently, beyond the histological analysis carried out by the anatomopathologist, there are only a few rare diagnostic tests based on molecular approaches. Microscopic observations of the morphology of the cells are supplemented in some laboratories by the search for certain protein markers with the aid of specific antibodies. These immunohistochemical tests nevertheless currently present a number of drawbacks. They deliver only a poor level of productivity (they only allow detection of a single antigen at a time), cannot distinguish without ambiguity between the different types of tumour cells, and only offer mediocre possibilities for quantitative detection of the markers. The diagnostic procedures performed on the basis of the detection of genetic markers (such as the heterozygote losses 1p and 19q in oligodendrogliomas) are currently less commonly used or are difficult to implement.

In recent years, scientists have discovered that a class of RNAs, referred to as miRNAs or microRNAs, can regulate gene expression both by degradation of target RNA messengers and by repression of their translation according to the degree of complementarity between the miRNA and its target RNAm.

MicroRNAs (miRNAs) represent a class of non-coding, endogenous, small (21 to 25 nt) single-strand RNAs identified relatively recently. The genes responsible for the expression of these miRNAs are transcribed beforehand in the form of long precursors, themselves cleaved into pre-miRNA by the RNAse III Drosha. These pre-miRNAs are matured by another RNAse, Dicer, which gives rise to a known RNA duplex [miRNA:miRNA*]. Only one of the two strands (the mature strand) contributes to the RNA-induced silencing complex so as to function as a post-transcriptional regulator (Bartel et al., Cell, 2004. 116(2): 281-97).

These RNAs play an important role in translation regulation and are therefore heavily involved in numerous cellular processes, such as development, differentiation, proliferation, apoptosis and the response to stress, these being processes that are often deregulated in tumours.

Numerous scientific works have been dedicated to the role of miRNAs in oncogenesis processes (Benard, J. and S. Douc-Rasy, Bull Cancer, 2005. 92(9): p. 757-62; Hammond Curr Opin Genet Dev, 2006. 16(1): p. 4-9). It has now been found that miRNAs are actively involved in the development of cancers.

In humans, the number of genes coding the currently identified precursors of miRNAs is 1048 (Sanger miRBase version 16, September 2010). Some of these miRNAs have been detected as tumour markers in the context of cancers of the breast and of the prostate and are therefore considered to be oncogenic miRNAs or tumour suppressors in accordance with their expression in the tumours and their post-transcriptional role.

In recent years, the potential alteration to expression levels of miRNAs in glial tumours has already been the subject of several scientific publications.

One publication by Ciafre et al. in 2005 (Biochem Biophys Res Commun. 2005. Sep. 9; 334(4): 1351-8) compares the expression levels of 245 miRNAs in glioblastomas and in normal tissues and demonstrates a clear expression profile of these miRNAs in glioblastomas compared to the expression profile in normal tissues. This publication describes the fact that miR-221 is overexpressed in glioblastomas compared to normal tissues, whereas miR-128, miR-181a, miR181b and miR181c are underexpressed in glioblastomas.

At the same time, Chen (N Engl J Med, 2005. 353 (17): 1768-71) describes the fact that miR-21 is highly overexpressed in glioblastomas compared to normal tissues.

Another investigation of expression levels of 192 miRNAs in glial tumour tissues and in normal tissues was carried out by Silber et al. in 2008 (BMC Med. 2008. Jun. 24; 6:14.). The aim of this study was to analyse a potential modification of the expression profile of miRNAs in anaplastic astrocytomas (grade III) or glioblastomas (grade IV) of tumours of different grades having the same cellular origins. This study shows a modified expression profile of miRNAs in anaplastic astrocytomas and glioblastomas compared to normal tissues. The miRNAs mentioned in this publication, of which the expression is modified, are not identical to those already mentioned in the earlier publications.

However, until now, no prior study has carried out an exhaustive investigation because never before have all microRNAs expressed in human cells been examined, and the prior art teaching also differs.

Furthermore, in the majority of cases, these studies only enable diagnosis of a glioma with respect to healthy tissue or peritumoral tissue, but do not make it possible to distinguish between a glioblastoma and an oligodendroglioma, these being two tumours of which the cellular origins and prognostics are very different and also require rather different treatments.

Although the study by Silber et al. (BMC Med. 2008. Jun. 24; 6:14.) showed that the expression profiles of miRNAs measured in anaplastic astrocytomas and those measured in glioblastomas are different, these tumours (grade III and grade IV respectively) are in fact tumours of the same nature, since they are both developed from astrocytes. In fact, anaplastic astrocytomas can develop quickly into a glioblastoma.

A method making it possible to distinguish between a glioblastomas (GBM) and an oligodendroglioma (ODG) provides an interest in terms of public health different from that of a method not enabling distinction between two high-grade tumours of identical nature. Then again, such a process cannot be generalised with regard to the mode of distinction of tumours of different nature.

In fact, none of these earlier studies makes it possible to accurately and quickly diagnose the presence of a glioblastoma or of an oligodendroglioma in a patient on the basis of the analysis of a group of biomarkers.

There is a great need to develop a diagnostic method capable of distinguishing between an oligodendroglioma and a glioblastoma, enabling a patient to receive care corresponding to his physical state and to increase his chances of survival.

The present invention relates to a method for in vitro diagnosis of a brain tumour belonging to the group formed by two types of tumours: ODGs and GBMs, and for identifying the type of tumour, said method comprising the following steps:

(i) measuring at least two ratios of expression levels of pairs of miRNAs extracted from a biological sample taken from a patient suspected of presenting one of the above-mentioned brain tumours, said two expression level ratios being, in particular:
1—a ratio of expression levels of an miRNA pair, said ratio being specific to ODGs, and
2—a ratio of expression levels of an miRNA pair, said ratio being specific to GBMs,

(ii) comparing the aforementioned ratios measured in said sample with those obtained respectively in a reference healthy tissue, a reference ODG tissue and a reference GBM tissue.

The present invention is based on the unexpected observation discovered by the inventors during an investigation intended to measure the expression levels of 847 human microRNAs in healthy brain tissues and in brain tumour tissues (GBM, ODG).

On the basis of this investigation, the inventors succeeded in identifying 23 miRNAs that constitute biological indicators (biomarkers) making it possible, thanks to the method described in the present invention, to distinguish between a healthy brain tissue, a tissue originating from a tumour of the glioblastoma type or a tissue originating from a tumour of the oligodendroglioma type.

The nucleotide sequences and also the accession numbers of 23 miRNAs are shown in Table 1 below.

Generally, the present invention is based on the dosage of the expression levels of the 23 microRNAs in a healthy brain tissue or a tissue originating from a tumour of the glioblastoma type or a tissue originating from a tumour of the oligodendroglioma type. The ratios of the expression levels of these microRNAs are then calculated per pair of microRNAs. The inventors have discovered that 21 ratios among those that can be calculated constitute specific signatures of a single tissue type or of two different tissue types (see example 1).

Thus, among these 21 ratios, the following are distinguished:
1. ratios that are specific to an ODG tumour tissue (ratios 1 and 2 in Table 2). The values of the ratios obtained for the ODG tissue are always at least 4 times greater than the values of these ratios calculated in a healthy brain tissue or a tissue originating from a tumour of the glioblastoma type,
2. ratios that are specific to a GBM tumour tissue (ratios 3 and 4 in Table 2). The values of the ratios obtained for the GBM tissue are always at least 4 times greater than the values of these ratios calculated in a healthy brain tissue or a tissue originating from a tumour of the oligodendroglioma type.
3. ratios that are specific to a glial tumour tissue (GBM or ODG) (ratios 5 to 10 in Table 2). The values of the ratios obtained for the glial tumour tissue are always at least 4 times greater than the values of these ratios calculated in a healthy brain tissue.
4. ratios that are specific to an ODG tumour tissue (ratios 11 to 18 in Table 2). The values of the ratios obtained for the ODG tissue are always at least 4 times greater than the values of these ratios calculated in a healthy brain tissue,
5. ratios that are specific to a GBM tumour tissue (ratios 19 to 21 in Table 2). The values of the ratios obtained for the GBM tissue are always at least 4 times greater than the values of these ratios calculated in a healthy brain tissue.

The presented invention is therefore based on the analysis of the expression levels of the microRNAs in a tissue suspected of being a tumour tissue, then on the calculation of the ratio values per microRNA pair and on a comparison of the values obtained for the ratios with those in a reference table (see example 2), which colligates the specific ratio values calculated for reference healthy tissues, reference GBM tissues and reference ODG tissues.

Thanks to the present invention, the analysis of the expression levels of microRNAs in a tissue suspected of being a tumour tissue makes it possible to specify the nature of the analysed tissue.
(i) If the values calculated for ratios 5 to 10 are significantly different from those of those ratios obtained for healthy tissues, but are close to those of the ratios for the GBMs or the ODGs, the tissue is confirmed as being a tumour tissue (without specification of the type of tumour).
(ii) If the values calculated for ratios 1 and 2 are significantly different from those of the ratios obtained for the healthy tissues and the GBMs, but are close to those of the ratios for the ODGs, the tissue is confirmed as being tumoral and presenting an ODG.
(iii) If the values calculated for ratios 3 and 4 are significantly different from those of those ratios obtained for the healthy tissues and the ODGs, but are close to those of the ratios for the GBMs, the tissue is confirmed as being tumoral and presenting a GBM.
(iv) If the values calculated for ratios 11 to 21 are significantly different from those of the ratios obtained for the healthy tissues and are close to those of the ratios for the ODGs, the tissue is confirmed as being tumoral and presenting an ODG (ratios 11 to 18), or if said values are significantly different from those of the ratios obtained for the healthy tissues and are close to those of the ratios for the GBMs, the tissue is confirmed as being tumoral and presenting a GBM (ratios 19 to 21).

In one embodiment of the invention, the present invention relates to a method for in vitro diagnosis of a brain tumour belonging to the group formed by two types of tumours: ODGs and GBMs, and for identifying the type of the tumour, said method comprising the following steps:
(i) measuring at least two ratios of expression levels of miRNA pairs extracted from a biological sample taken from a patient suspected of presenting one of the above-mentioned brain tumours, said two expression level ratios being:
   a ratio no. 1 of the expression levels of miRNA pairs selected from group 1 formed by hsa-miR-106a/hsa-miR-494 and hsa-miR-17/hsa-miR-494,
   a ratio no. 2 of the expression levels of miRNA pairs selected from group 2 formed by hsa-miR-210/hsa-miR-193b and hsa-miR-210/hsa-miR-423-5p,
(ii) comparing the above-mentioned ratios measured in said sample with those obtained respectively in a reference healthy tissue, a reference ODG tissue and a reference GBM tissue.

The miRNAs used in the present invention are referenced in the miRBase database (http://www.mirbase.org/) (miRBase: tools for microRNA genomic, Griffiths-Jones et al., NAR 2008 36(Database Issue):D154-D158) where the nucleic sequence of an miRNA can be found by an accession number MIMAT or a uniform annotation. An annotation system has been put in place in order to name the different miRNAs (A uniform system for microRNA annotation, Ambros et al., RNA 2003 9(3):277-279). In this system, the microRNAs are denoted by a number. This identification number is preceded by the abbreviation “miR” or “mir”, which makes it possible to distinguish between the mature microRNA (miR) and the precursor loop (hairpin) of the microRNA (mir), corresponding respectively to a MIMAT accession number and an MI accession number. A prefix of three or four letters is used to distinguish the genomic origins of this miRNA. For example, “hsa-miR-126” means that this mature miRNA originates from the human genome. However, the mature microRNAs can be very close (different by only one base). In this case, the microRNAs bear the same number and are distinguished from one another by a lower case letter, such as a, b, c . . . for example hsa-miR-26b. However, a precursor loop may generate, from its 5′ side and its 3′ side, two mature microRNAs that are distinguished from one another by the annotation “5p” or “3p”. For example, “hsa-miR-151-3p” means a mature miRNA originating from the 3′ side of its precursor. However, a precursor loop of the microRNA can derive two mature miRNAs, that is to say a major product and a minor product. Such a minor miRNA is distinguished from the major miRNA derived from the same precursor by a star followed by an identification number, for example “hsa-miR-425*” or hsa-miR-425-star.

In the present invention, the expressions “miRNA” and “microRNA” can be replaced by one another. The expressions “glial tumours” or “gliomas” are equivalent. Glioblastomas and oligodendrogliomas are two types of gliomas. A “healthy tissue” or “healthy sample” mean non-tumoral tissue or non-tumoral sample. These expressions in the singular can include the plural and vice versa.

The nucleic acid sequences of the miRNAs mentioned above and used in the present invention as well as their accession numbers are presented in Example 1 (Table 1).

TABLE 1

| Names | | | Sanger |
|---|---|---|---|
| of the | Sequence | | miRBase |
| miRNAs | no. | Sequence | accession no. |
| hsa-miR-320c | SEQ ID NO: 1 | AAAAGCUGGGUUGAGAGGGU | MIMAT0005793 |
| hsa-miR-320a | SEQ ID NO: 2 | AAAAGCUGGGUUGAGAGGGCGA | MIMAT0000510 |
| hsa-miR-320b | SEQ ID NO: 3 | AAAAGCUGGGUUGAGAGGGCAA | MIMAT0005792 |
| hsa-miR-491-5p | SEQ ID NO: 4 | AGUGGGGAACCCUUCCAUGAGG | MIMAT0002807 |
| hsa-miR-127-3p | SEQ ID NO: 5 | UCGGAUCCGUCUGAGCUUGGCU | MIMAT0000446 |
| hsa-miR-132 | SEQ ID NO: 6 | UAACAGUCUACAGCCAUGGUCG | MIMAT0000426 |
| hsa-miR-574-3p | SEQ ID NO: 7 | CACGCUCAUGCACACACCCACA | MIMAT0003239 |
| hsa-miR-494 | SEQ ID NO: 8 | UGAAACAUACACGGGAAACCUC | MIMAT0002816 |
| hsa-miR-107 | SEQ ID NO: 9 | AGCAGCAUUGUACAGGGCUAUCA | MIMAT0000104 |
| hsa-miR-23b | SEQ ID NO: 10 | AUCACAUUGCCAGGGAUUACC | MIMAT0000418 |
| hsa-miR-425-star | SEQ ID NO: 11 | AUCGGGAAUGUCGUGUCCGCCC | MIMAT0003393 |
| hsa-miR-345 | SEQ ID NO: 12 | GCUGACUCCUAGUCCAGGGCUC | MIMAT0000772 |
| hsa-miR-423-5p | SEQ ID NO: 13 | UGAGGGGCAGAGAGCGAGACUUU | MIMAT0004748 |
| hsa-miR-193b | SEQ ID NO: 14 | AACUGGCCCUCAAAGUCCCGCU | MIMAT0002819 |
| hsa-miR-320d | SEQ ID NO: 15 | AAAAGCUGGGUUGAGAGGA | MIMAT0006764 |
| hsa-miR-1207-5p | SEQ ID NO: 16 | UGGCAGGGAGGCUGGGAGGGG | MIMAT0005871 |
| hsa-miR-210 | SEQ ID NO: 17 | CUGUGCGUGUGACAGCGGCUGA | MIMAT0000267 |
| hsa-miR-92a | SEQ ID NO: 18 | UAUUGCACUUGUCCCGGCCUGU | MIMAT0000092 |
| hsa-miR-106a | SEQ ID NO: 19 | AAAAGUGCUUACAGUGCAGGUAG | MIMAT0000103 |
| hsa-miR-17 | SEQ ID NO: 20 | CAAAGUGCUUACAGUGCAGGUAG | MIMAT0000070 |
| hsa-miR-191 | SEQ ID NO: 21 | CAACGGAAUCCCAAAAGCAGCUG | MIMAT0000440 |
| hsa-miR-324-5p | SEQ ID NO: 22 | CGCAUCCCCUAGGGCAUUGGUGU | MIMAT0000761 |
| hsa-miR-185 | SEQ ID NO: 23 | UGGAGAGAAAGGCAGUUCCUGA | MIMAT0000455 |

The expression levels of the miRNAs can be measured by any conventional method, such as
“DNA-chip” hybridisation,
high-rate sequencing methods of a large number of individualised miRNAs,
real-time PCR,
Northern blot,
   or else by any other method specific to miRNAs.

The 23 microRNAs can be measured individually and independently or simultaneously without altering the analysis procedure described by the present invention.

The expression level of the miRNAs can be measured by the “DNA-chip” technique. The “DNA-chip” technique is well known to a person skilled in the art. It involves the hybridisation of extracted miRNAs on a solid support formed by a nylon membrane, a silicon or glass surface, possibly nanobeads or particles, comprising oligonucleotides of known sequences fixed on the support or adhering thereto. The complementarity of the fixed oligonucleotides with the sequences of the microRNAs or their conversion products (amplification products, cDNA, RNA or cRNA) makes it possible to generate a signal (fluorescence, luminescence, radioactivity, electric signal . . . ) in accordance with the employed labelling techniques at the oligonucleotides immobilised on the supports (DNA chips). This signal is detected by specific equipment and an intensity value of this signal is thus recorded for each miRNA. Several types of chips designed for detection of miRNAs are already available on the market, such as the miRNA GeneChip® marketed by Affymetrix, miRcury arrays by Exiqon, and miRXplore microarrays by Miltenyi.

In the case of high-rate analysis by sequencing, the miRNAs are extracted and purified from a sample, are isolated from one another by methods proposed by the manufacturers of sequencing equipment, such as Roche, Invitrogen. This genre of analysis consists in individualising the molecules of the different microRNAs, in proceeding with an amplification step, and in sequencing the products (“nucleic acid clones”) thus generated. The implementation of a very large number of sequences in order to identify each of these “clones” (several thousands) makes it possible to generate a listing of the microRNAs present in a sample and to quantify each of these miRNAs by quite simply counting the number of times each sequence is found in the detailed listing.

The expression “biological sample” means a tissue sample, in particular a brain tissue obtained by exeresis. The sample can be frozen or fixed in paraffin (tissue fragment fixed by chemical treatment and enclosed in a block of paraffin in accordance with conventional anatomopathology techniques). The biological sample may also be a sample of biological liquids, in particular a blood sample (in non-coagulated form or derivatives thereof, that is to say plasma or serum).

A brain tissue sample may be a tissue of the central nervous system originating from the cerebral cortex, the cerebellar cortex, the basal nuclei or the brainstem nuclei.

If “a biological sample originating from a patient suspected of presenting one of the above-mentioned brain tumours” is a brain tissue sample, it is a tissue removed, via biopsy or via exeresis by means of surgery or any other suitable method of intratumoral removal, from the suspected tumoral location in a patient. The suspected tumoral location is identified beforehand by means of a conventional method, such as MRI, a PET scan (scanner using a gamma ray image), or a conventional scanner.

If “a biological sample originating from a patient suspected of presenting one of the above-mentioned brain tumours” is a blood sample, this sample is taken from a patient by removing blood in accordance with the conventional methods.

“A reference healthy tissue” may be a brain tissue devoid of any tumoral nature according to a histological analysis conducted by anatomopathologists. The reference healthy tissue may be a tissue from the central nervous system originating from the cerebral cortex, the cerebellar cortex, the basal nuclei or the brainstem nuclei. The reference healthy tissue is preferably removed from the same patient (healthy tissue from the area surrounding the tumour). Alternatively, it may be formed by encephalic fragments obtained after “corticectomy” surgery performed on individuals not presenting brain tumours but within the scope of intervention for treatment of epilepsy or following head injuries.

“A reference healthy tissue” may also be a sample of biological liquids, in particular a blood serum or a blood plasma taken from a healthy person, and in particular from a person not presenting any type of cancer.

“A reference ODG tissue” is a tissue originating from a patient in which the presence of oligodendrogliomas has been certified by anatomopathalogical analysis.

“A reference GBM tissue” is a tissue originating from a patient in which the presence of glioblastomas has been diagnosed by anatomopathalogical analysis.

“A reference ODG tissue” or “a reference GBM tissue” can be a glial tumour fragment obtained by exeresis in a patient and of which the tumour type has be certified by anatomopathalogical analysis.

Ideally, the diagnosis of oligodendrogliomas or glioblastomas can also be confirmed by molecular analyses, such as analysis of the gene expression profiles of these tumoral tissues. The tumours are then characterised by using the classification method specified by Li et al (Li A, Walling J, Ahn S, Kotliarov Y, Su Q, Quezado M, Oberholtzer J C, Park J, Zenklusen J C, Fine H A. Cancer Res. 2009 Mar. 1; 69(5):2091-9), which is based on the use of expression levels of a set of 54 genes. The glioblastomas or oligodendrogliomas are classified respectively into groups G and O of the above-cited publication.

A ratio of the expression levels of two microRNAs measured in a sample is considered as significantly different from a corresponding ratio established beforehand in a reference tissue if the ratio obtained in said sample is 4 times greater or 4 times less than the ratio established beforehand in said reference tissue.

By contrast, a ratio obtained in a sample is not considered different from a corresponding ratio established beforehand in a reference tissue if the ratio obtained in said sample is not 4 times greater or 4 times less than the ratio established beforehand in said reference tissue.

The ratio of the expression levels of an miRNA pair belonging to group 1 obtained from an ODG tissue is at least 4 times greater than that obtained from a healthy tissue or that obtained from a GBM tissue, whereas this ratio is not different from the ratio obtained from a reference ODG tissue.

By contrast, the ratio of the expression levels of an miRNA pair belonging to group 2 obtained from a GBM tissue is at least 4 times greater than that obtained from a healthy tissue or that obtained from a ODG tissue, whereas this ratio is not different from the ratio obtained from a reference GBM tissue.

In a specific embodiment, the presence of ODG in the sample originating from a patient suspected of presenting one of the above-mentioned tumours is deduced
if the ratio no. 1 measured in said sample is not at least 4 times greater or at least 4 times less than that measured in a reference ODG tissue or if the ratio no. 2 measured in said sample is at least 4 times less than that obtained in a reference GBM tissue, and
if the ratio no. 1 measured in said sample is at least 4 times greater than that measured in a reference healthy tissue and than that obtained in a reference GBM tissue.

In another specific embodiment, the presence of GBM in the sample originating from a patient suspected of presenting one of the above-mentioned tumours is deduced
if the ratio no. 2 measured in said sample is not at least 4 times greater or at least 4 times less than that obtained in a reference GBM tissue or if the ratio no. 1 measured in said sample is at least 4 times less than that obtained in a reference ODG tissue, and
if the ratio no. 2 measured in said sample is at least 4 times greater than that obtained in a reference healthy tissue and than that obtained in a reference ODG tissue.

In an advantageous embodiment, the in vitro diagnostic method according to the invention also comprises the measurement of a ratio of group 3 of expression levels of miRNA pairs extracted from a biological sample originating from a patient suspected of presenting one of the above-mentioned brain tumours, said ratio being the ratio of the expression levels of pairs selected from hsa-miR-423-5p/hsa-miR-491-5p, hsa-miR-210/hsa-miR-491-5p, hsa-miR-92a/hsa-miR-127-3p, hsa-miR-92a/hsa-miR-132, hsa-miR-320a/hsa-miR-491-5p and hsa-miR-320c/hsa-miR-491-5p.

This advantageous embodiment is based on the fact that the inventors, in addition to groups 1 and 2 of miRNA pairs, have also been successful in identifying a group 3 of miRNA pairs of which the expression level ratios in gliomas, in other words oligodendrogliomas and glioblastomas, are significantly different from those measured in a healthy tissue.

The ratio of the expression levels of such an miRNA pair, that is to say hsa-miR-423-5p/hsa-miR-491-5p, hsa-miR-210/hsa-miR-491-5p, hsa-miR-92a/hsa-miR-127-3p, hsa-miR-92a/hsa-miR-132, hsa-miR-320a/hsa-miR-491-5p and hsa-miR-320c/hsa-miR-491-5p, obtained in an ODG tissue or a GBM tissue, is at least 4 times greater than that obtained in a healthy tissue.

Consequently, this ratio obtained from a sample originating from a patient makes it possible to indicate the possible presence of a glioma in the aforementioned sample.

In a specific embodiment, the above-mentioned method according to the invention comprises the following steps:
(i) measuring three ratios of the expression levels of miRNA pairs extracted from a sample originating from a patient suspected of presenting one of the above-mentioned brain tumours, said three expression level ratios being:
   ratio no. 3 of the expression levels of pairs selected from hsa-miR-423-5p/hsa-miR-491-5p, hsa-miR-210/hsa-miR-491-5p, hsa-miR-92a/hsa-miR-127-3p, hsa-miR-92a/hsa-miR-132, hsa-miR-320a/hsa-miR-491-5p and hsa-miR-320c/hsa-miR-491-5p,
   ratio no. 1 of the expression levels of pairs selected from hsa-miR-106a/hsa-miR-494 and hsa-miR-17/hsa-miR-494,
   ratio no. 2 of the expression levels of pairs selected from hsa-miR-210/hsa-miR-193b and hsa-miR-210/hsa-miR-423-5p,
(ii) comparing the above-mentioned ratios obtained in said sample with those obtained respectively in a reference healthy tissue, a reference ODG tissue and a reference GBM tissue.

In a specific embodiment, the presence of ODG in the sample originating from a patient suspected of presenting one of the above-mentioned tumours is deduced
if ratio no. 1 measured in said sample is not at least 4 times greater or at least 4 times less than that obtained in a reference ODG tissue or if ratio no. 2 measured in said sample is at least 4 times less than that obtained in a reference GBM sample, and
if ratio no. 1 measured in said sample is at least 4 times greater than that measured in a reference healthy tissue and than that obtained in a reference GBM tissue, and
if ratio no. 3 measured in said sample is at least 4 times greater than that obtained in a reference healthy tissue.

In a further specific embodiment, the presence of GBM in the sample originating from a patient suspected of presenting one of the above-mentioned tumours is deduced
if ratio no. 2 measured in said sample is not at least 4 times greater or at least 4 times less than that obtained in a reference GBM tissue or if ratio no. 1 measured in said sample is at least 4 times less than that obtained in a reference ODG tissue, and
if ratio no. 2 measured in said sample is at least 4 times greater than that obtained in a reference healthy tissue and than that obtained in a reference ODG tissue, and
if ratio no. 3 measured in said sample is at least 4 times greater than that obtained in a reference healthy tissue.

In an advantageous embodiment, the in vitro diagnostic method according to the invention also comprises the measurement of a ratio of group 4 of expression levels of miRNA pairs extracted from a biological sample originating from a patient suspected of presenting one of the above-mentioned brain tumours, said ratio being the ratio of the expression levels of pairs selected from hsa-miR-320a/hsa-miR-127-3p, hsa-miR-320b/hsa-miR-127-3p, hsa-miR-320c/hsa-miR-127-3p, hsa-miR-17/hsa-miR-107, hsa-miR-106a/hsa-miR-23b, hsa-miR-17/hsa-miR-23b, hsa-miR-345/hsa-miR-574-3p, and hsa-miR-191/hsa-miR-425-star.

The value of a ratio of group 4 obtained for ODG tissue is always at least 4 times greater than the values of this ratio calculated in healthy brain tissue. This value makes it possible to indicate the possible presence of ODG tissue in a sample originating from a patient suspected of presenting a glial tumour.

In a specific embodiment, the above-mentioned method according to the invention comprises the following steps:
(i) measuring four ratios of the expression levels of miRNA pairs extracted from a sample originating from a patient suspected of presenting one of the above-mentioned brain tumours, said four expression level ratios being:
   ratio no. 3 of the expression levels of pairs selected from hsa-miR-423-5p/hsa-miR-491-5p, hsa-miR-210/hsa-miR-491-5p, hsa-miR-92a/hsa-miR-127-3p, hsa-miR-92a/hsa-miR-132, hsa-miR-320a/hsa-miR-491-5p and hsa-miR-320c/hsa-miR-491-5p,
   ratio no. 1 of the expression levels of pairs selected from hsa-miR-106a/hsa-miR-494 and hsa-miR-17/hsa-miR-494,
   ratio no. 2 of the expression levels of pairs selected from hsa-miR-210/hsa-miR-193b and hsa-miR-210/hsa-miR-423-5p,
   ratio no. 4 of the expression levels of pairs selected from hsa-miR-320a/hsa-miR-127-3p, hsa-miR-320b/hsa-miR-127-3p, hsa-miR-320c/hsa-miR-127-3p, hsa-miR-17/hsa-miR-107, hsa-miR-106a/hsa-miR-23b, hsa-miR-17/hsa-miR-23b, hsa-miR-345/hsa-miR-574-3p, and hsa-miR-191/hsa-miR-425-star.

In this specific embodiment, the presence of ODG in the sample originating from a patient suspected of presenting one of the above-mentioned tumours is confirmed:
if ratio no. 1 measured in said sample is not at least 4 times greater or at least 4 times less than that obtained in a reference ODG tissue or if ratio no. 2 measured in said sample is at least 4 times less than that obtained in a reference GBM tissue, and
if ratio no. 1 measured in said sample is at least 4 times greater than that measured in a reference healthy tissue and than that obtained in a reference GBM tissue, and
if ratio no. 3 measured in said sample is at least 4 times greater than that obtained in a reference healthy tissue, and
if ratio no. 4 measured in said sample is at least 4 times greater than that measured in a reference healthy tissue.

In another advantageous embodiment, the in vitro diagnostic method according to the invention also comprises the measurement of a ratio of group 5 of expression levels of miRNA pairs extracted from a biological sample originating from a patient suspected of presenting one of the above-mentioned brain tumours, said ratio being the ratio of the expression levels of pairs selected from hsa-miR-345/hsa-miR-324-5p, hsa-miR-345/hsa-miR-185 and hsa-miR-345/hsa-miR-320d.

The value of a ratio of group 5 obtained for GBM tissue is always at least 4 times greater than the values of this ratio calculated in a healthy brain tissue, this value makes it possible to indicate the possible presence of GBM tissue in a sample originating from a patient suspected of presenting a glial tumour.

In a specific embodiment, the above-mentioned method according to the invention comprises the following steps:
(i) measuring four ratios of the expression levels of miRNA pairs extracted from a sample originating from a patient suspected of presenting one of the above-mentioned brain tumours, said four expression level ratios being:
   ratio no. 3 of the expression levels of pairs selected from hsa-miR-423-5p/hsa-miR-491-5p, hsa-miR-210/hsa-miR-491-5p, hsa-miR-92a/hsa-miR-127-3p, hsa-miR-92a/hsa-miR-132, hsa-miR-320a/hsa-miR-491-5p and hsa-miR-320c/hsa-miR-491-5p,
   ratio no. 1 of the expression levels of pairs selected from hsa-miR-106a/hsa-miR-494 and hsa-miR-17/hsa-miR-494,
   ratio no. 2 of the expression levels of pairs selected from hsa-miR-210/hsa-miR-193b and hsa-miR-210/hsa-miR-423-5p,
   ratio no. 5 of the expression levels of pairs selected from hsa-miR-345/hsa-miR-324-5p, hsa-miR-345/hsa-miR-185 and hsa-miR-345/hsa-miR-320d,
(ii) comparing the above-mentioned ratios obtained in said sample with those obtained respectively in a reference healthy tissue, a reference ODG tissue and a reference GBM tissue.

In this specific embodiment, the presence of GBM in the sample originating from a patient suspected of presenting one of the above-mentioned tumours is confirmed:
if ratio no. 2 measured in said sample is not at least 4 times greater or at least 4 times less than that obtained in a reference GBM tissue or if the ratio of group 1 measured in said sample is at least 4 times less than that obtained in a reference ODG tissue, and
if ratio no. 2 measured in said sample is at least 4 times greater than that obtained in a reference healthy tissue and than that obtained in a reference ODG tissue, and
if ratio no. 3 measured in said sample is at least 4 times greater than that obtained in a reference healthy tissue,
if ratio no. 5 measured in said sample is at least 4 times greater than that measured in a reference healthy tissue.

In a particularly advantageous embodiment of the invention, the in vitro diagnostic method also comprises the measurement in a biological sample originating from a patient suspected of presenting one of the above-mentioned brain tumours:
of a ratio no. 4 of the expression levels of miRNA pairs selected from group 4 formed by hsa-miR-320a/hsa-miR-127-3p, hsa-miR-320b/hsa-miR-127-3p, hsa-miR-320c/hsa-miR-127-3p, hsa-miR-17/hsa-miR-107, hsa-miR-106a/hsa-miR-23b, hsa-miR-17/hsa-miR-23b, hsa-miR-345/hsa-miR-574-3p, and hsa-miR-191/hsa-miR-425-star, and
of a ratio no. 5 of the expression levels of miRNA pairs selected from group 5 formed by hsa-miR-345/hsa-miR-324-5p, hsa-miR-345/hsa-miR-185 and hsa-miR-345/hsa-miR-320d.

In a specific embodiment, the above-mentioned method according to the invention comprises the following steps:

(i) measuring five ratios of the expression levels of miRNA pairs extracted from a sample originating from a patient suspected of presenting one of the above mentioned brain tumours, said five expression level ratios being:
ratio no. 3 of the expression levels of pairs selected from hsa-miR-423-5p/hsa-miR-491-5p, hsa-miR-210/hsa-miR-491-5p, hsa-miR-92a/hsa-miR-127-3p, hsa-miR-92a/hsa-miR-132, hsa-miR-320a/hsa-miR-491-5p and hsa-miR-320c/hsa-miR-491-5p,
ratio no. 1 of the expression levels of pairs selected from hsa-miR-106a/hsa-miR-494 and hsa-miR-17/hsa-miR-494,
ratio no. 2 of the expression levels of pairs selected from hsa-miR-210/hsa-miR-193b and hsa-miR-210/hsa-miR-423-5p,
ratio no. 4 of the expression levels of pairs selected from hsa-miR-320a/hsa-miR-127-3p, hsa-miR-320b/hsa-miR-127-3p, hsa-miR-320c/hsa-miR-127-3p, hsa-miR-17/hsa-miR-107, hsa-miR-106a/hsa-miR-23b, hsa-miR-17/hsa-miR-23b, hsa-miR-345/hsa-miR-574-3p, and hsa-miR-191/hsa-miR-425-star, and
ratio no. 5 of the expression levels of pairs selected from hsa-miR-345/hsa-miR-324-5p, hsa-miR-345/hsa-miR-185 and hsa-miR-345/hsa-miR-320d,

(ii) comparing the above-mentioned ratios obtained in said sample with those obtained respectively in a reference healthy tissue, a reference ODG tissue and a reference GBM tissue.

In this specific embodiment, the presence of ODG in the sample originating from a patient suspected of presenting one of the above-mentioned tumours is deduced:
if ratio no. 1 measured in said sample is not at least 4 times greater or at least 4 times less than that obtained in a reference ODG tissue or if ratio no. 2 measured in said sample is at least 4 times less than that obtained in a reference GBM tissue, and
if ratio no. 1 measured in said sample is at least 4 times greater than that measured in a reference healthy tissue and than that obtained in a reference GBM tissue, and
if ratio no. 3 measured in said sample is at least 4 times greater than that obtained in a reference healthy tissue, and
if ratio no. 4 measured in said sample is at least 4 times greater than that measured in a reference healthy tissue.

By contrast, the presence of GBM in the sample originating from a patient suspected of presenting one of the above-mentioned tumours is deduced:
if ratio no. 2 measured in said sample is not at least 4 times greater or at least 4 times less than that obtained in a reference GBM tissue or if the ratio of group 1 measured in said sample is at least 4 times less than that obtained in a reference ODG tissue, and
if ratio no. 2 measured in said sample is at least 4 times greater than that obtained in a reference healthy tissue and than that obtained in a reference ODG tissue, and
if ratio no. 3 measured in said sample is at least 4 times greater than that obtained in a reference healthy tissue,
if ratio no. 5 measured in said sample is at least 4 times greater than that measured in a reference healthy tissue.

The present invention is illustrated by way of example by the following examples. These examples are not in any case intended to limit the potential embodiment of the invention.

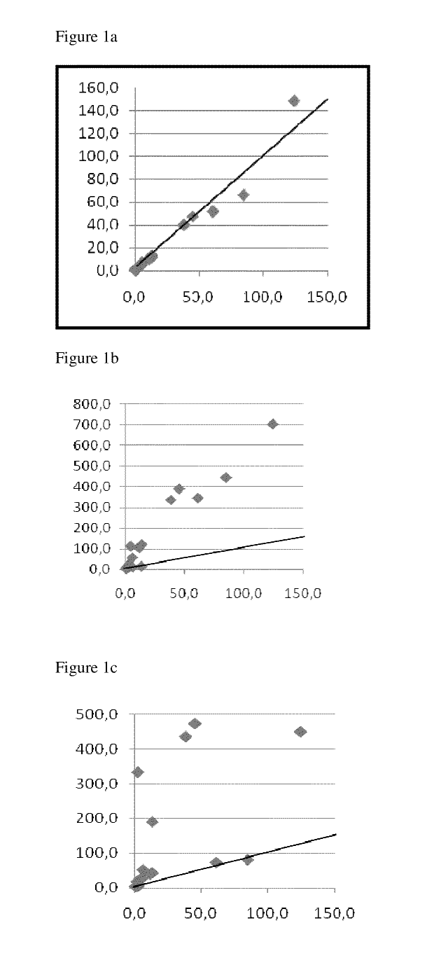

FIG. 1a: FIG. 1a illustrates the comparison of the values of the 21 ratios of the microRNA pairs dosed in the suspected sample A (shown in Table 3), these values being shown on the ordinate, with the values of the ratios in a reference healthy tissue (values in Table 2), these values being shown on the abscissa. The ratio values for the suspected sample A are correlated with the respective values for a reference healthy tissue. The straight line shown in the graph illustrates the theoretical situation of perfect correlation between the values obtained for the suspected sample A and those obtained for the reference sample.

FIG. 1b: FIG. 1b illustrates the comparison of the values of the 21 ratios of the microRNA pairs dosed in the suspected sample A (shown in Table 3), these values being shown on the ordinate, with the values of the ratios in a reference ODG tissue (values in Table 2), these values being shown on the abscissa. The ratio values for the suspected sample A are correlated with the respective values for a reference ODG tissue. The straight line shown in the graph illustrates the theoretical situation of perfect correlation between the values obtained for the suspected sample A and those obtained for the reference sample.

FIG. 1c: FIG. 1c illustrates the comparison of the values of the 21 ratios of the microRNA pairs dosed in the suspected sample A (shown in Table 3), these values being shown on the ordinate, with the values of the ratios in a reference GBM tissue (values in Table 2), these values being shown on the abscissa. The ratio values for the suspected sample A are correlated with the respective values for a reference GBM tissue. The straight line shown in the graph illustrates the theoretical situation of perfect correlation between the values obtained for the suspected sample A and those obtained for the reference sample.

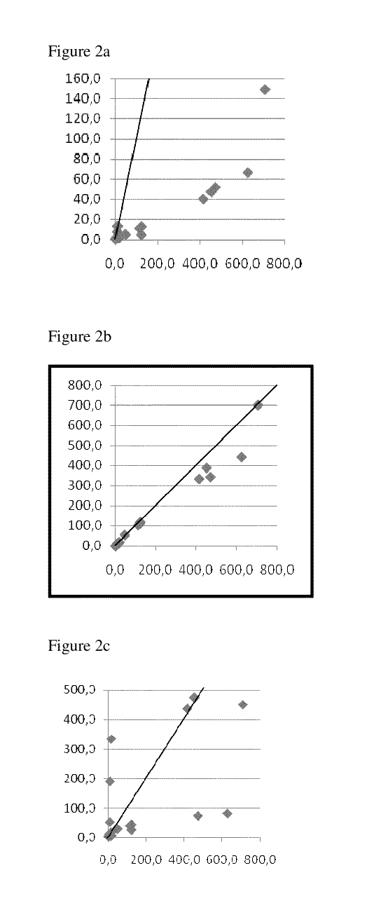

FIG. 2a: FIG. 2a illustrates the comparison of the values of the 21 ratios of the microRNA pairs dosed in the suspected sample B (shown in Table 3), these values being shown on the ordinate, with the values of the ratios in a reference healthy tissue (values in Table 2), these values being shown on the abscissa. The ratio values for the suspected sample B are correlated with the respective values for a reference healthy tissue. The straight line shown in the graph illustrates the theoretical situation of perfect correlation between the values obtained for the suspected sample B and those obtained for the reference sample.

FIG. 2b: FIG. 2b illustrates the comparison of the values of the 21 ratios of the microRNA pairs dosed in the suspected sample B (shown in Table 3), these values being shown on the ordinate, with the values of the ratios in a reference ODG tissue (values in Table 2), these values being shown on the abscissa. The ratio values for the suspected sample B are correlated with the respective values for a reference ODG tissue. The straight line shown in the graph illustrates the theoretical situation of perfect correlation between the values obtained for the suspected sample B and those obtained for the reference sample.

FIG. 2c: FIG. 2c illustrates the comparison of the values of the 21 ratios of the microRNA pairs dosed in the suspected sample B (shown in Table 3), these values being shown on the ordinate, with the values of the ratios in a reference GBM tissue (values in Table 2), these values being shown on the abscissa. The ratio values for the suspected sample B are correlated with the respective values for a reference GBM tissue. The straight line shown in the graph illustrates the theoretical situation of perfect correlation between the values obtained for the suspected sample B and those obtained for the reference sample.

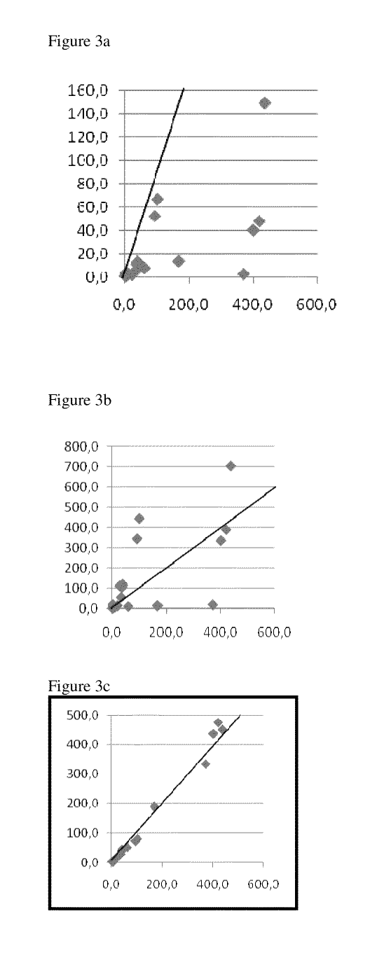

FIG. 3a: FIG. 3a illustrates the comparison of the values of the 21 ratios of the microRNA pairs dosed in the suspected sample C (shown in Table 3), these values being shown on the ordinate, with the values of the ratios in a reference healthy tissue (values in Table 2), these values being shown on the abscissa. The ratio values for the suspected sample C are correlated with the respective values for a reference healthy tissue. The straight line shown in the graph illustrates the theoretical situation of perfect correlation between the values obtained for the suspected sample C and those obtained for the reference sample.

FIG. 3b: FIG. 3b illustrates the comparison of the values of the 21 ratios of the microRNA pairs dosed in the suspected sample C (shown in Table 3), these values being shown on the ordinate, with the values of the ratios in a reference ODG tissue (values in Table 2), these values being shown on the abscissa. The ratio values for the suspected sample C are correlated with the respective values for a reference ODG tissue. The straight line shown in the graph illustrates the theoretical situation of perfect correlation between the values obtained for the suspected sample C and those obtained for the reference sample.

FIG. 3c: FIG. 3c illustrates the comparison of the values of the 21 ratios of the microRNA pairs dosed in the suspected sample C (shown in Table 3), these values being shown on the ordinate, with the values of the ratios in a reference GBM tissue (values in Table 2), these values being shown on the abscissa. The ratio values for the suspected sample C are correlated with the respective values for a reference GBM tissue. The straight line shown in the graph illustrates the theoretical situation of perfect correlation between the values obtained for the suspected sample C and those obtained for the reference sample.

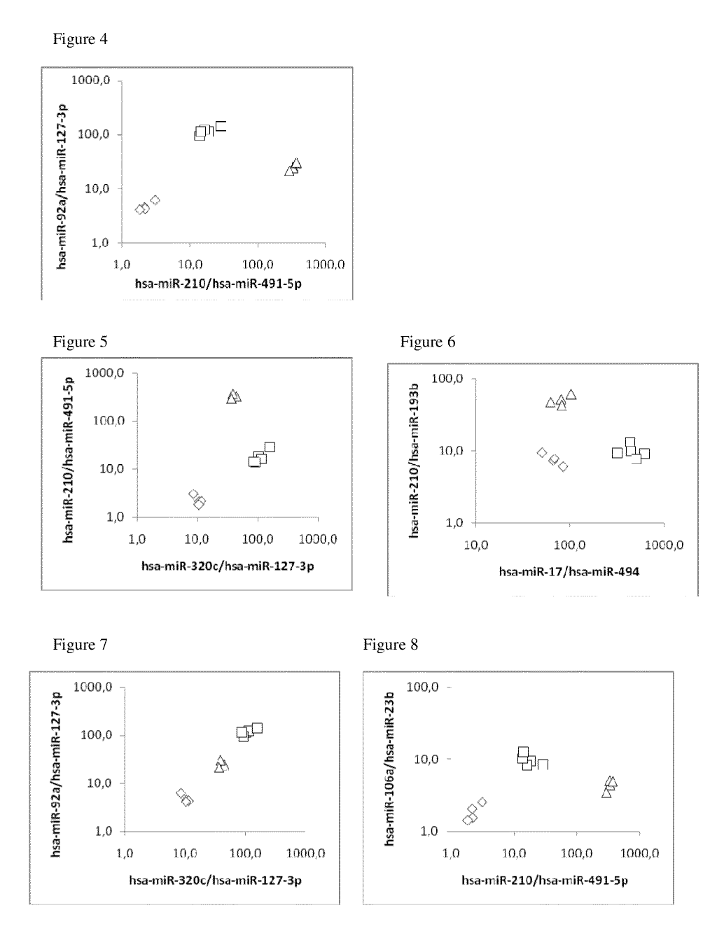

FIG. 4: FIG. 4 shows a two-dimensional graph, in which the values of the ratio hsa-miR-210/hsa-miR-491-5p measured in the different samples are shown on the abscissa; and the values of the ratio hsa-miR-92a/hsa-miR-127-3p measured in these samples are shown on the ordinate. The diamonds represent the healthy tissue samples. The squares represent the ODG samples. The triangles represent the GBM samples.

FIG. 5: FIG. 5 shows a two-dimensional graph, in which the values of the ratio hsa-miR-320c/hsa-miR-127-3p measured in the different samples are shown on the abscissa; and the values of the ratio hsa-miR-210/hsa-miR-491-5p measured in these samples are shown on the ordinate. The diamonds represent the healthy tissue samples. The squares represent the ODG samples. The triangles represent the GBM samples.

FIG. 6: FIG. 6 shows a two-dimensional graph, in which the values of the ratio hsa-miR-17/hsa-miR-494 measured in the different samples are shown on the abscissa; and the values of the ratio hsa-miR-210/hsa-miR-193b measured in these samples are shown on the ordinate. The diamonds represent the healthy tissue samples. The squares represent the ODG samples. The triangles represent the GBM samples.

FIG. 7: FIG. 7 shows a two-dimensional graph, in which the values of the ratio hsa-miR-320c/hsa-miR-127-3p measured in the different samples are shown on the abscissa; and the values of the ratio hsa-miR-92a/hsa-miR-127-3p measured in these samples are shown on the ordinate. The diamonds represent the healthy tissue samples. The squares represent the ODG samples. The triangles represent the GBM samples.

FIG. 8: FIG. 8 shows a two-dimensional graph, in which the values of the ratio hsa-miR-210/hsa-miR-491-5p measured in the different samples are shown on the abscissa; and the values of the ratio hsa-miR-106a/hsa-miR-23b measured in these samples are shown on the ordinate. The diamonds represent the healthy tissue samples. The squares represent the ODG samples. The triangles represent the GBM samples.

### EXAMPLE 1

### Material and Methods

1.1 Tumoral Samples

The tumoral samples of glioblastomas, oligodendrogliomas and meningiomas and also the samples of healthy tissue (corticectomies) were obtained after exeresis by neurosurgeons in the surgery department of Grenoble university hospital centre and were immediately frozen at −80° C. Tissue sections approximately 40 μm thick were then produced with the aid of a cryotome in sufficient number to obtain approximately 80 mg of tissue and were stored at −80° C. until extraction of the RNAs.

1.2 Biological Liquids

For purification of the microRNAs circulating in the blood, blood samples were taken from the patients using sampling tubes of the PAXgene Blood RNA type (PreAnalytix-Qiagen—BD company). The circulating cells were lysed completely and the RNAs were collected by centrifugation and purified in accordance with the manufacturer's instructions.

With regard to the microRNA fractions contained in the microvesicles produced by the tumours and present in the blood, the method used is directly based on that described by Skog et coll (Skog J, Würdinger T, van Rijn S, Meijer D H, Gainche L, Sena-Esteves M, Curry W T Jr, Carter B S, Krichevsky A M, Breakefield X O. Glioblastoma microvesicles transport RNA and proteins that promote tumour growth and provide diagnostic biomarkers. (Skog et coll, Nat Cell Biol. 2008 December; 10(12):1470-6. Epub 2008 Nov. 16)).

The microvesicles were purified from the sera by centrifugation and microfiltration. The sera were centrifuged a first time for 10 min at 300 g, then the supernatants were then centrifuged for 20 min at 17,000 g and filtered over a 0.22 micron filter. The microvesicles were obtained by ultracentrifugation of the filtrate at 110,000 g for 70 min. The pellet was then resuspended in phosphate buffer (PBS) and the RNAs were extracted in accordance with the protocol described above.

1.3 RNA Extraction

The RNAs were extracted with use of the hsa-miRVana™ kit (Ambion, ABI), making it possible to separate the long RNAs (size >250 nt) and the short RNAs (size <250 nt) by following the manufacturers recommendations. The tissue was then first lysed briefly, the RNAs were then precipitated after adding sodium acetate and ethanol, extracted in the presence of a phenol/chloroform mixture, were separated in accordance with their size on chromatographic columns, and were then eluted. The eluted RNAs were then quantified by measuring the OD at 260 nm with the aid of the Nanodrop ND-1000 spectrophotometer, and the quality of these RNAs was checked by means of electrophoretic migration in a polymer gel using a BioAnalyser 2100 with use of the RNA 6000 nano LabChip® kit (Agilent).

1.4 Dosage of the miRNAs by DNA-Chip Hybridisation

The short RNAs (size less than 250 bases) obtained during the proceeding step were used for DNA-chip hybridisation (GeneChip miRNA Affymetrix).

The first step consists in adding a poly-A end in position 3′ of the RNAs. The second step consists in labelling the RNAs. For this purpose, a ligation reaction will be carried out in order to add biotinylated DNA to the RNAs (for example with the labelling and detection reagents from the Flash Tag HSR kit from Genisphere, Hatfield, USA). Once this step has been performed, hybridisation will be able to occur with the probes located on the chip. After hybridisation, there is a step of amplification of the labelling, then washing steps, which are carried out before the chip is scanned. The chip will then be scanned and an image of each chip that will have to be treated in order to obtain an intensity value corresponding to the RNA/probe hybridisation for each microRNA is obtained. The data are then processed and standardised using QC Tools software in accordance with the methods proposed by Irizarry et al (R A. Irizarry, B Hobbs, F Collin, Y D. Beazer-Barclay, K J. Antonellis, U Scherf and T P. Speed. Exploration, Normalization, and Summaries of High Density Oligonucleotide Array Probe Level Data. Biostatistics, April 2003; Vol. 4; Number 2: 249-264).

Alternatively, the results can also be generated by RT-Q-PCR dosage, any other type of DNA chip from any manufacturer, or any other dosage method. The analyses can be carried out on the biological liquids (blood or subfraction for example).

1.5 Dosage of the miRNAs by Real-Time PCR

The expression of miRNAs is quantified by means of quantitative PCR with the aid of the kits distributed by Applied Biosystems, specific to mature miRNAs. In a first step, 80 ng of short RNAs are inversely transcribed (into single-stranded cDNA) in the presence of loop primers, which impart the specificity for the quantification of the expression of the mature miRNAs. The real-time PCR is then carried out with the aid of the primers provided in the kits. One of the primers comprises fluorescent groups (probe referred to as Taqman@), which makes it possible to perform a quantitative measurement using a suitable fluorimeter, such as the Stratagene Mx3005. The detection threshold is determined in a first step by the user at the start of the exponential phase. The Ct value corresponds to the number of cycles from which the fluorescence exceeds this detection threshold. This Ct value is proportional to the quantity of cDNA initially present in the sample. The calculation of the ratios between two given miRNAs from Ct measurements taken for the same sample and for two different miRNAs (referred to here for example as miR_{A} and miR_{B}) is obtained using the following formula: hsa-miR_{A}/hsa-miR_{B}=2̂(Ct hsa-miR_{A}-Ct hsa-miR_{B})

### EXAMPLE 2

A chart of the ratios of the expression levels of miRNA pairs (Table 2 below) is determined from the ratios measured in a reference healthy tissue (N), a reference ODG tissue (ODG), and a reference GBM tissue (GBM).

The reference healthy tissue, the reference ODG tissue and the reference GBM tissue were obtained as described as in Example 1 (part 1.1).

The expression levels of the miRNAs were measured in accordance with the method described in Example 1. In order to increase readability and so as to provide ratio values greater than 1 in the majority of cases, the calculated values have been multiplied by a factor of 10.

TABLE 2

| | | | |
|---|---|---|---|
| ratio | N | ODG | GBM |

group 1: glioma type: ODG

| | | | | |
|---|---|---|---|---|
| 1 | hsa-miR-106a/hsa-miR-494 | 51.9 | 344.4 | 72.9 |
| 2 | hsa-miR-17/hsa-miR-494 | 66.3 | 443.7 | 80.8 |

group 2: glioma type: GBM

| | | | | |
|---|---|---|---|---|
| 3 | hsa-miR-210/hsa-miR-193b | 7.3 | 9.9 | 51.7 |
| 4 | hsa-miR-210/hsa-miR-423-5p | 13.0 | 13.5 | 190.4 |

group 3: glioma markers

| | | | | |
|---|---|---|---|---|
| 5 | hsa-miR-423-5p/hsa-miR-491-5p | 1.7 | 13.6 | 17.5 |
| 6 | hsa-miR-210/hsa-miR-491-5p | 2.2 | 18.4 | 334.1 |
| 7 | hsa-miR-92a/hsa-miR-127-3p | 4.5 | 111.7 | 25.7 |
| 8 | hsa-miR-92a/hsa-miR-132 | 4.9 | 55.1 | 29.0 |
| 9 | hsa-miR-320a/hsa-miR-491-5p | 47.5 | 389.1 | 474.3 |
| 10 | hsa-miR-320c/hsa-miR-491-5p | 39.9 | 335.1 | 436.7 |

group 4: glioma markers (ODG)

| | | | | |
|---|---|---|---|---|
| 11 | hsa-miR-320a/hsa-miR-127-3p | 12.6 | 120.8 | 42.8 |
| 12 | hsa-miR-320b/hsa-miR-127-3p | 12.1 | 115.3 | 43.1 |
| 13 | hsa-miR-320c/hsa-miR-127-3p | 10.6 | 104.0 | 39.4 |
| 14 | hsa-miR-17/hsa-miR-107 | 1.7 | 7.3 | 5.2 |
| 15 | hsa-miR-106a/hsa-miR-23b | 2.1 | 9.5 | 4.4 |
| 16 | hsa-miR-17/hsa-miR-23b | 2.6 | 12.3 | 4.9 |
| 17 | hsa-miR-345/hsa-miR-574-3p | 3.2 | 20.1 | 5.3 |
| 18 | hsa-miR-191/hsa-miR-425-star | 148.8 | 702.4 | 450.7 |

group 5: glioma markers (GBM)

| | | | | |
|---|---|---|---|---|
| 19 | hsa-miR-345/hsa-miR-324-5p | 2.3 | 6.8 | 10.6 |
| 20 | hsa-miR-345/hsa-miR-185 | 0.6 | 1.6 | 3.7 |
| 21 | hsa-miR-345/hsa-miR-320d | 0.8 | 1.9 | 3.6 |

The use of the reference values established in the chart (Table 2) within the scope of the execution of analyses in order to characterise a sample suspected of being tumoral is illustrated in Examples 3 to 5 hereinafter.

### EXAMPLE 3

Three different samples (A, B or C) were analysed in Example 3. The respective rates of each of the 23 mirRNAs from Table 1 were measured in these samples. The values of the 21 ratios described in Table 2 between microRNAs dosed in the same sample were then calculated. The results obtained are shown in Table 3. It can be seen by comparison of the values in Table 3 with those indicated in Table 2 that the ratio values in sample A are very close to the ratio values characteristic of a normal sample. It can be concluded that sample A is a normal brain tissue. The respective microRNA ratio values for sample B are very similar to the values characteristic of ODGs. Sample B is therefore diagnosed as ODG. The respective microRNA ratio values for sample C are very similar to the values characteristic of GBMs. Sample C is therefore diagnosed as GBM.

TABLE 3

| | ratio | A | B | C |
|---|---|---|---|---|
| 1 | hsa-miR-106a/hsa-miR-494 | 61.1 | 471.6 | 94.0 |
| 2 | hsa-miR-17/hsa-miR-494 | 84.9 | 625.6 | 102.6 |
| 3 | hsa-miR-210/hsa-miR-193b | 6.0 | 9.0 | 61.4 |
| 4 | hsa-miR-210/hsa-miR-423-5p | 13.3 | 9.9 | 169.0 |
| 5 | hsa-miR-423-5p/hsa-miR-491-5p | 1.6 | 16.5 | 22.0 |
| 6 | hsa-miR-210/hsa-miR-491-5p | 2.2 | 16.4 | 371.8 |
| 7 | hsa-miR-92a/hsa-miR-127-3p | 4.3 | 122.7 | 30.4 |
| 8 | hsa-miR-92a/hsa-miR-132 | 5.6 | 48.0 | 35.5 |
| 9 | hsa-miR-320a/hsa-miR-491-5p | 45.3 | 452.2 | 420.9 |
| 10 | hsa-miR-320c/hsa-miR-491-5p | 38.4 | 415.5 | 401.3 |
| 11 | hsa-miR-320a/hsa-miR-127-3p | 13.6 | 123.7 | 40.4 |
| 12 | hsa-miR-320b/hsa-miR-127-3p | 12.9 | 121.3 | 40.5 |
| 13 | hsa-miR-320c/hsa-miR-127-3p | 11.5 | 113.7 | 38.5 |
| 14 | hsa-miR-17/hsa-miR-107 | 1.2 | 8.6 | 5.5 |
| 15 | hsa-miR-106a/hsa-miR-23b | 1.5 | 8.2 | 4.9 |
| 16 | hsa-miR-17/hsa-miR-23b | 2.1 | 10.9 | 5.4 |
| 17 | hsa-miR-345/hsa-miR-574-3p | 2.6 | 20.4 | 4.9 |
| 18 | hsa-miR-191/hsa-miR-425-star | 124.4 | 706.5 | 437.8 |
| 19 | hsa-miR-345/hsa-miR-324-5p | 2.6 | 5.7 | 12.0 |
| 20 | hsa-miR-345/hsa-miR-185 | 0.6 | 1.2 | 4.1 |
| 21 | hsa-miR-345/hsa-miR-320d | 0.8 | 2.0 | 40.1 |

### EXAMPLE 4

In Example 4, the examination was carried out identically to that indicated in Example 3 above. However, in order to make the interpretation of the analyses more direct and clear, a representation of the results obtained in graph form, this being preferred by the inventors, is proposed by way of example. The values of the ratios between the microRNA pairs dosed in the same sample (shown in Table 3 for samples A, B and C) are shown in two-dimensional graphs (FIGS. 1a, 1b, 1c for sample A, FIGS. 2a, 2b, 2c for sample B, and FIGS. 3a, 3b, 3c for sample C), in which the ratio values of the reference samples (values in Table 2) are shown on the abscissa and the identical ratio values obtained for the analysed samples (Table 3) are shown on the ordinate. The ratio values for a suspected sample are correlated with the respective values for a reference healthy tissue, a reference ODG tissue and a reference GBM tissue. For each of the graphs, the straight line (y=x) shown in the graphs illustrates the theoretical perfect correlation between the values obtained for a suspected sample and those for a reference sample. The greater the number of points of a graph positioned close to the straight line of perfect correlation in the graph, the stronger is the identity of the analysed tissue with the reference tissue under consideration in said graph. Looking at the graphs in FIG. 1, it can be seen that sample A is very similar to the reference healthy tissue and is clearly distinguished from the ODG or GBM tissues. It is therefore deduced that sample A is a healthy brain tissue. Similarly, it can be concluded that sample B is an ODG tissue and that sample C is a GBM tissue.

### EXAMPLE 5

In example 5, the values of two ratios between microRNAs in the same sample are used to create a graph that makes it possible to distinguish very quickly between the types of tissues. In these graphs, and for each tissue sample, the value obtained for a given ratio between a microRNA pair is shown on the abscissa and the values for a second ratio for another microRNA pair are shown on the ordinate. Each sample is represented by a point on the graph. This graph makes it possible to establish, as shown by FIGS. 4-8, clouds of points grouped consistently according to the type of tissue. The healthy tissues thus form a set of coordinate points rather separate from glial tumours, and the ODG tissues are grouped in a coordinate zone rather separate from the cloud of points corresponding to GBM tissues. For any unknown sample, it is therefore possible to ascertain on the basis of the values of a ratio pair between miRNAs, by means of this graph, whether the sample shows characteristics identical to those of a healthy tissue, an ODG tissue or a GBM tissue, and therefore to deduce the nature of this analysed sample.

1. An in vitro diagnostic method for diagnosing a brain tumour belonging to the group formed by 2 types of tumours ODGs and GBMs, and for identifying the type of tumour, said method comprising the following steps:
(i) measuring at least two ratios of expression levels of miRNA pairs extracted from a biological sample taken from a patient suspected of presenting one of the above-mentioned brain tumours, said two expression level ratios being:
a ratio no. 1 of the expression levels of miRNA pairs selected from group 1 formed by hsa-miR-106a/hsa-miR-494 and hsa-miR-17/hsa-miR-494,
a ratio no. 2 of the expression levels of miRNA pairs selected from group 2 formed by hsa-miR-210/hsa-miR-193b and hsa-miR-210/hsa-miR-423-5p,
(ii) comparing the above-mentioned ratios measured in said sample with those obtained respectively in a reference healthy tissue, a reference ODG tissue and a reference GBM tissue.

2. The method according to claim 1, wherein the presence of ODG in the sample originating from a patient suspected of presenting one of the above-mentioned tumours is deduced
if the ratio no. 1 measured in said sample is not at least 4 times greater or at least 4 times less than that measured in a reference ODG tissue or if the ratio no. 2 measured in said sample is at least 4 times less than that obtained in a reference GBM tissue, and
if the ratio no. 1 measured in said sample is at least 4 times greater than that measured in a reference healthy tissue and than that obtained in a reference GBM tissue.

3. The method according to claim 1, wherein the presence of GBM in the sample originating from a patient suspected of presenting one of the above-mentioned tumours is deduced
if the ratio no. 2 measured in said sample is not at least 4 times greater or at least 4 times less than that obtained in a reference GBM tissue or if the ratio no. 1 measured in said sample is at least 4 times less than that obtained in a reference ODG tissue, and
if the ratio no. 2 measured in said sample is at least 4 times greater than that obtained in a reference healthy tissue and than that obtained in a reference ODG tissue.

4. The method according to claim 1, further comprising the measurement of a ratio no. 3 of the expression levels of miRNA pairs in a biological sample originating from a patient suspected of presenting one of the above-mentioned brain tumours, said ratio being the ratio of the expression levels of pairs selected from hsa-miR-423-5p/hsa-miR-491-5p, hsa-miR-210/hsa-miR-491-5p, hsa-miR-92a/hsa-miR-127-3p, hsa-miR-92a/hsa-miR-132, hsa-miR-320a/hsa-miR-491-5p and hsa-miR-320c/hsa-miR-491-5p.

5. The method according to claim 4, comprising the following steps:
(i) measuring three ratios of the expression levels of miRNA pairs extracted from a sample originating from a patient suspected of presenting one of the above-mentioned brain tumours, said three expression level ratios being:
ratio no. 3 of the expression levels of pairs selected from hsa-miR-423-5p/hsa-miR-491-5p, hsa-miR-210/hsa-miR-491-5p, hsa-miR-92a/hsa-miR-127-3p, hsa-miR-92a/hsa-miR-132, hsa-miR-320a/hsa-miR-491-5p and hsa-miR-320c/hsa-miR-491-5p,
ratio no. 1 of the expression levels of pairs selected from hsa-miR-106a/hsa-miR-494 and hsa-miR-17/hsa-miR-494,
ratio no. 2 of the expression levels of pairs selected from hsa-miR-210/hsa-miR-193b and hsa-miR-210/hsa-miR-423-5p,
(ii) comparing the above-mentioned ratios obtained in said sample with those obtained respectively in a reference healthy tissue, a reference ODG tissue and a reference GBM tissue.

6. The method according to claim 5, wherein the presence of ODG in the sample originating from a patient suspected of presenting one of the above-mentioned tumours is deduced
if ratio no. 1 measured in said sample is not at least 4 times greater or at least 4 times less than that obtained in a reference ODG tissue or if ratio no. 2 measured in said sample is at least 4 times less than that obtained in a reference GBM sample, and
if ratio no. 1 measured in said sample is at least 4 times greater than that measured in a reference healthy tissue and than that obtained in a reference GBM tissue, and
if ratio no. 3 measured in said sample is at least 4 times greater than that obtained in a reference healthy tissue.

7. The method according to claim 5, wherein the presence of GBM in the sample originating from a patient suspected of presenting one of the above-mentioned tumours is deduced
if ratio no. 2 measured in said sample is not at least 4 times greater or at least 4 times less than that obtained in a reference GBM tissue or if ratio no. 1 measured in said sample is at least 4 times less than that obtained in a reference ODG tissue, and
if ratio no. 2 measured in said sample is at least 4 times greater than that obtained in a reference healthy tissue and than that obtained in a reference ODG tissue, and
if ratio no. 3 measured in said sample is at least 4 times greater than that obtained in a reference healthy tissue.

8. The method according to claim 4, further comprising the measurement in a biological sample originating from a patient suspected of presenting one of the above-mentioned brain tumours
of a ratio no. 4 of the expression levels of miRNA pairs selected from group 4 formed by hsa-miR-320a/hsa-miR-127-3p, hsa-miR-320b/hsa-miR-127-3p, hsa-miR-320c/hsa-miR-127-3p, hsa-miR-17/hsa-miR-107, hsa-miR-106a/hsa-miR-23b, hsa-miR-17/hsa-miR-23b, hsa-miR-345/hsa-miR-574-3p, and hsa-miR-191/hsa-miR-425-star.

9. The method according to claim 8, said method comprising the following step:
(i) measuring four ratios of the expression levels of miRNA pairs extracted from a sample originating from a patient suspected of presenting one of the above-mentioned brain tumours, said four expression level ratios being:
ratio no. 3 of the expression levels of pairs selected from hsa-miR-423-5p/hsa-miR-491-5p, hsa-miR-210/hsa-miR-491-5p, hsa-miR-92a/hsa-miR-127-3p, hsa-miR-92a/hsa-miR-132, hsa-miR-320a/hsa-miR-491-5p and hsa-miR-320c/hsa-miR-491-5p,
ratio no. 1 of the expression levels of pairs selected from hsa-miR-106a/hsa-miR-494 and hsa-miR-17/hsa-miR-494,
ratio no. 2 of the expression levels of pairs selected from hsa-miR-210/hsa-miR-193b and hsa-miR-210/hsa-miR-423-5p,
ratio no. 4 of the expression levels of pairs selected from hsa-miR-320a/hsa-miR-127-3p, hsa-miR-320b/hsa-miR-127-3p, hsa-miR-320c/hsa-miR-127-3p, hsa-miR-17/hsa-miR-107, hsa-miR-106a/hsa-miR-23b, hsa-miR-17/hsa-miR-23b, hsa-miR-345/hsa-miR-574-3p, and hsa-miR-191/hsa-miR-425-star.

10. The method according to claim 9, wherein the presence of ODG in the sample originating from a patient suspected of presenting one of the above-mentioned tumours is confirmed:
if ratio no. 1 measured in said sample is not at least 4 times greater or at least 4 times less than that obtained in a reference ODG tissue or if ratio no. 2 measured in said sample is at least 4 times less than that obtained in a reference GBM tissue, and
if ratio no. 1 measured in said sample is at least 4 times greater than that measured in a reference healthy tissue and than that obtained in a reference GBM tissue, and
if ratio no. 3 measured in said sample is at least 4 times greater than that obtained in a reference healthy tissue, and
if ratio no. 4 measured in said sample is at least 4 times greater than that measured in a reference healthy tissue.

11. The method according to claim 4, further comprising the measurement in a biological sample originating from a patient suspected of presenting one of the above-mentioned brain tumours of a ratio no. 5 of the expression levels of miRNA pairs selected from group 5 formed by hsa-miR-345/hsa-miR-324-5p, hsa-miR-345/hsa-miR-185 and hsa-miR-345/hsa-miR-320d.

12. The method according to claim 11, said method comprising the following steps:
(i) measuring four ratios of the expression levels of miRNA pairs extracted from a sample originating from a patient suspected of presenting one of the above-mentioned brain tumours, said four expression level ratios being:
ratio no. 3 of the expression levels of pairs selected from hsa-miR-423-5p/hsa-miR-491-5p, hsa-miR-210/hsa-miR-491-5p, hsa-miR-92a/hsa-miR-127-3p, hsa-miR-92a/hsa-miR-132, hsa-miR-320a/hsa-miR-491-5p and hsa-miR-320c/hsa-miR-491-5p,
ratio no. 1 of the expression levels of pairs selected from hsa-miR-106a/hsa-miR-494 and hsa-miR-17/hsa-miR-494,
ratio no. 2 of the expression levels of pairs selected from hsa-miR-210/hsa-miR-193b and hsa-miR-210/hsa-miR-423-5p,
ratio no. 5 of the expression levels of pairs selected from hsa-miR-345/hsa-miR-324-5p, hsa-miR-345/hsa-miR-185 and hsa-miR-345/hsa-miR-320d,
(ii) comparing the above-mentioned ratios obtained in said sample with those obtained respectively in a reference healthy tissue, a reference ODG tissue and a reference GBM tissue.

13. The method according to claim 12, wherein the presence of GBM in the sample originating from a patient suspected of presenting one of the above-mentioned tumours is confirmed:
if ratio no. 2 measured in said sample is not at least 4 times greater or at least 4 times less than that obtained in a reference GBM tissue or if the ratio of group 1 measured in said sample is at least 4 times less than that obtained in a reference ODG tissue, and
if ratio no. 2 measured in said sample is at least 4 times greater than that obtained in a reference healthy tissue and than that obtained in a reference ODG tissue, and
if ratio no. 3 measured in said sample is at least 4 times greater than that obtained in a reference healthy tissue,
if ratio no. 5 measured in said sample is at least 4 times greater than that measured in a reference healthy tissue.

14. The method according to claim 4, further comprising the measurement in a biological sample originating from a patient suspected of presenting one of the above-mentioned brain tumours
of a ratio no. 4 of the expression levels of miRNA pairs selected from group 4 formed by hsa-miR-320a/hsa-miR-127-3p, hsa-miR-320b/hsa-miR-127-3p, hsa-miR-320c/hsa-miR-127-3p, hsa-miR-17/hsa-miR-107, hsa-miR-106a/hsa-miR-23b, hsa-miR-17/hsa-miR-23b, hsa-miR-345/hsa-miR-574-3p, and hsa-miR-191/hsa-miR-425-star, and
of a ratio no. 5 of the expression levels of miRNA pairs selected from group 5 formed by hsa-miR-345/hsa-miR-324-5p, hsa-miR-345/hsa-miR-185 and hsa-miR-345/hsa-miR-320d.

15. The method according to claim 14, said method comprising the following steps:
(i) measuring five ratios of the expression levels of miRNA pairs extracted from a sample originating from a patient suspected of presenting one of the above mentioned brain tumours, said five expression level ratios being:
ratio no. 3 of the expression levels of pairs selected from hsa-miR-423-5p/hsa-miR-491-5p, hsa-miR-210/hsa-miR-491-5p, hsa-miR-92a/hsa-miR-127-3p, hsa-miR-92a/hsa-miR-132, hsa-miR-320a/hsa-miR-491-5p and hsa-miR-320c/hsa-miR-491-5p,
ratio no. 1 of the expression levels of pairs selected from hsa-miR-106a/hsa-miR-494 and hsa-miR-17/hsa-miR-494,
ratio no. 2 of the expression levels of pairs selected from hsa-miR-210/hsa-miR-193b and hsa-miR-210/hsa-miR-423-5p,
ratio no. 4 of the expression levels of pairs selected from hsa-miR-320a/hsa-miR-127-3p, hsa-miR-320b/hsa-miR-127-3p, hsa-miR-320c/hsa-miR-127-3p, hsa-miR-17/hsa-miR-107, hsa-miR-106a/hsa-miR-23b, hsa-miR-17/hsa-miR-23b, hsa-miR-345/hsa-miR-574-3p, and hsa-miR-191/hsa-miR-425-star, and
ratio no. 5 of the expression levels of pairs selected from hsa-miR-345/hsa-miR-324-5p, hsa-miR-345/hsa-miR-185 and hsa-miR-345/hsa-miR-320d,
(ii) comparing the above-mentioned ratios obtained in said sample with those obtained respectively in a reference healthy tissue, a reference ODG tissue and a reference GBM tissue.

16. The method according to claim 15, wherein
the presence of ODG in the sample originating from a patient suspected of presenting one of the above-mentioned tumours is deduced:
if ratio no. 1 measured in said sample is not at least 4 times greater or at least 4 times less than that obtained in a reference ODG tissue or if ratio no. 2 measured in said sample is at least 4 times less than that obtained in a reference GBM tissue, and
if ratio no. 1 measured in said sample is at least 4 times greater than that measured in a reference healthy tissue and than that obtained in a reference GBM tissue, and
if ratio no. 3 measured in said sample is at least 4 times greater than that obtained in a reference healthy tissue, and
if ratio no. 4 measured in said sample is at least 4 times greater than that measured in a reference healthy tissue.

17. The method according to claim 15, wherein the presence of GBM in the sample originating from a patient suspected of presenting one of the above-mentioned tumours is deduced:
if ratio no. 2 measured in said sample is not at least 4 times greater or at least 4 times less than that obtained in a reference GBM tissue or if the ratio of group 1 measured in said sample is at least 4 times less than that obtained in a reference ODG tissue, and
if ratio no. 2 measured in said sample is at least 4 times greater than that obtained in a reference healthy tissue and than that obtained in a reference ODG tissue, and
if ratio no. 3 measured in said sample is at least 4 times greater than that obtained in a reference healthy tissue,
if ratio no. 5 measured in said sample is at least 4 times greater than that measured in a reference healthy tissue.

18. The method according to claim 5, further comprising the measurement in a biological sample originating from a patient suspected of presenting one of the above-mentioned brain tumours
of a ratio no. 4 of the expression levels of miRNA pairs selected from group 4 formed by hsa-miR-320a/hsa-miR-127-3p, hsa-miR-320b/hsa-miR-127-3p, hsa-miR-320c/hsa-miR-127-3p, hsa-miR-17/hsa-miR-107, hsa-miR-106a/hsa-miR-23b, hsa-miR-17/hsa-miR-23b, hsa-miR-345/hsa-miR-574-3p, and hsa-miR-191/hsa-miR-425-star.

19. The method according to claim 5, further comprising the measurement in a biological sample originating from a patient suspected of presenting one of the above-mentioned brain tumours of a ratio no. 5 of the expression levels of miRNA pairs selected from group 5 formed by hsa-miR-345/hsa-miR-324-5p, hsa-miR-345/hsa-miR-185 and hsa-miR-345/hsa-miR-320d.

20. The method according to claim 5, further comprising the measurement in a biological sample originating from a patient suspected of presenting one of the above-mentioned brain tumours
of a ratio no. 4 of the expression levels of miRNA pairs selected from group 4 formed by hsa-miR-320a/hsa-miR-127-3p, hsa-miR-320b/hsa-miR-127-3p, hsa-miR-320c/hsa-miR-127-3p, hsa-miR-17/hsa-miR-107, hsa-miR-106a/hsa-miR-23b, hsa-miR-17/hsa-miR-23b, hsa-miR-345/hsa-miR-574-3p, and hsa-miR-191/hsa-miR-425-star, and
of a ratio no. 5 of the expression levels of miRNA pairs selected from group 5 formed by hsa-miR-345/hsa-miR-324-5p, hsa-miR-345/hsa-miR-185 and hsa-miR-345/hsa-miR-320d.


SEQUENCE LISTING (23 sequences)

SEQ ID NO: 1 - RNA, 20 nt, Homo sapiens
aaaagcuggguugagagggu

SEQ ID NO: 2 - RNA, 22 nt, Homo sapiens
aaaagcuggguugagagggcga

SEQ ID NO: 3 - RNA, 22 nt, Homo sapiens
aaaagcuggguugagagggcaa

SEQ ID NO: 4 - RNA, 22 nt, Homo sapiens
aguggggaacccuuccaugagg

SEQ ID NO: 5 - RNA, 22 nt, Homo sapiens
ucggauccgucugagcuuggcu

SEQ ID NO: 6 - RNA, 22 nt, Homo sapiens
uaacagucuacagccauggucg

SEQ ID NO: 7 - RNA, 22 nt, Homo sapiens
cacgcucaugcacacacccaca

SEQ ID NO: 8 - RNA, 22 nt, Homo sapiens
ugaaacauacacgggaaaccuc

SEQ ID NO: 9 - RNA, 23 nt, Homo sapiens
agcagcauuguacagggcuauca

SEQ ID NO: 10 - RNA, 21 nt, Homo sapiens
aucacauugccagggauuacc

SEQ ID NO: 11 - RNA, 22 nt, Homo sapiens
aucgggaaugucguguccgccc

SEQ ID NO: 12 - RNA, 22 nt, Homo sapiens
gcugacuccuaguccagggcuc

SEQ ID NO: 13 - RNA, 23 nt, Homo sapiens
ugaggggcagagagcgagacuuu

SEQ ID NO: 14 - RNA, 22 nt, Homo sapiens
aacuggcccucaaagucccgcu

SEQ ID NO: 15 - RNA, 19 nt, Homo sapiens
aaaagcuggguugagagga

SEQ ID NO: 16 - RNA, 21 nt, Homo sapiens
uggcagggaggcugggagggg

SEQ ID NO: 17 - RNA, 22 nt, Homo sapiens
cugugcgugugacagcggcuga

SEQ ID NO: 18 - RNA, 22 nt, Homo sapiens
uauugcacuugucccggccugu

SEQ ID NO: 19 - RNA, 23 nt, Homo sapiens
aaaagugcuuacagugcagguag

SEQ ID NO: 20 - RNA, 23 nt, Homo sapiens
caaagugcuuacagugcagguag

SEQ ID NO: 21 - RNA, 23 nt, Homo sapiens
caacggaaucccaaaagcagcug

SEQ ID NO: 22 - RNA, 23 nt, Homo sapiens
cgcauccccuagggcauuggugu

SEQ ID NO: 23 - RNA, 22 nt, Homo sapiens
uggagagaaaggcaguuccuga